(19) United States
(12) Patent Application Publication
(10) Pub. No.: US 2026/0363557 A1
(43) Pub. Date: Nov. 19, 2026

(54) INTEGRATED MULTI-USE PUMP SYSTEM

(71) Applicant: KONINKLIJKE PHILIPS N.V., EINDHOVEN (NL)

(72) Inventors: DIMITRI GEORGE KOSTAKIS, ORLANDO, FL (US); AVRAHAM YEINI, ORLANDO, FL (US)

(21) Appl. No.: 19/138,709

(22) PCT Filed: Nov. 29, 2023
(86) PCT No.: PCT/EP2023/083601

§ 371 Date: Jun. 13, 2025

Related U.S. Application Data
(60) Provisional application No. 63/432,069, filed on Dec. 13, 2022.

(51) Int. Cl. A61B 5/0205; A61B 5/021; A61B 5/022; A61B 5/083; A61B 5/097

(52) U.S. Cl. CPC A61B 5/0205; A61B 5/02141; A61B 5/022; A61B 5/0836; A61B 5/097

(57) ABSTRACT

An integrated multi-use pump system includes at least one pump and a flow path. The flow path flows air to a first subsystem that requires a first flow rate and a first pressure, and to a second subsystem that requires a second flow rate different than the first flow rate and that requires a second pressure different than the first pressure.

### BACKGROUND

Different fluid pump systems require different flow rates at different pressures. For example, non-invasive blood pressure requires pushing air to a high flow rate (0.6 L/min) and a high pressure (300 mmHg), whereas capnography requires pulling air at a lower flow rate (60 mL/min) at ambient pressure. Capnography involves measuring partial pressure of CO2 from an airway. Different fluid pump systems are designed separately, which results in space volume constraints and increased power consumption.

### SUMMARY

According to an aspect of the present disclosure, an integrated multi-use pump system includes at least one pump and a flow path. The flow path flows air to a first subsystem that requires a first flow rate and a first pressure, and to a second subsystem that requires a second flow rate different than the first flow rate and that requires a second pressure different than the first pressure.

According to another aspect of the present disclosure, a method of operating an integrated multi-use pump system includes receiving and filtering exhaled air; stepping down a flow rate of the exhaled air; mixing the exhaled air with ambient air to obtain an air mixture; filtering the air mixture; passing the air mixture through a check valve; inputting the air mixture to a pump; and applying pressure to a pressure to a pressure volume using the air mixture.

According to yet another aspect of the present disclosure, an integrated multi-use pump system includes at least one pump and a conduit. The conduit has a diameter that varies along a flow path so as to provide the flow path with a first flow rate and a first pressure for air flowing to a first subsystem on a first side of the conduit in the flow path and so as to provide the flow path with a second flow rate and a second pressure for air flowing to a second subsystem on a second side of the conduit in the flow path opposite to the first side of the conduit in the flow path.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates an integrated multi-use pump system, in accordance with a representative embodiment.

FIG. 2 illustrates another integrated multi-use pump system, in accordance with a representative embodiment.

FIG. 3 illustrates another integrated multi-use pump system, in accordance with a representative embodiment.

FIG. 4 illustrates a method of operation for an integrated multi-use pump system, in accordance with another representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, two or more subsystems with varying requirements for pressure and flow rate may be integrated in a system with central pumping, while still being independently operable. The integration of the two or more different fluid systems with a single pump drive system may result in space constraints and a reduction in power consumption by components such as pumps and drive circuitry. As a result, the same pump drive system may be used to integrate multiple subsystems such as non-invasive blood pressure (NBP) pushing air and CO2 pulling air, using various flow reduction methods throughout and using the same air flow path, to reduce total power consumption and save space within a system.

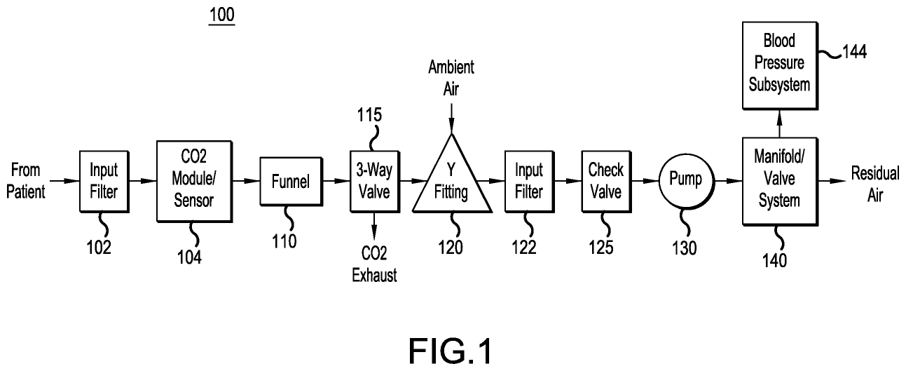

FIG. 1 illustrates an integrated multi-use pump system, in accordance with a representative embodiment.

The system 100 includes an input filter 102, a CO2 module/sensor 104, a funnel 110, a 3-way valve 115, a Y fitting 120, an input filter 122, a check valve 125, a pump 130, a manifold-valve system 140, and a blood pressure subsystem 144.

The system 100 in FIG. 1 is simplified, and includes the CO2 module/sensor 104 as a capnography module requiring patient exhaled air to be pulled through a chamber for CO2 measurement. This air is pulled through the CO2 module/sensor 104 via the pump 130 on the far side of the system 100. At the same time, the blood pressure subsystem 144 may include a non-invasive blood pressure cuff which requires air to be pushed into a volume to inflate the blood pressure cuff. The pump 130 is used to push air to the blood pressure subsystem 144 and to pull air for the CO2 module/sensor 104.

The system 100 includes the input filter 102 between the patient and the CO2 module/sensor 104 to filter the patient exhaled air. The input filter 102 may comprise a first input filter. The input filter 102 passes filtered air to the CO2 module/sensor 104. The CO2 module/sensor 104 is an example of a first subsystem, and specifically comprises a capnography subsystem in embodiments based on FIG. 1. The CO2 module/sensor 104 may require a low flow rate of 60 mL/min and ambient pressure.

The CO2 module/sensor 104 outputs air through a funnel 110. The funnel 110 is an example of a flow rate step down. The flow rate output of the air from the CO2 module/sensor 104 is stepped up from the left of the funnel 110 to the right of the funnel 110 in FIG. 1. The diameter of the funnel 110 is stepped down through the funnel 110 from left to right in FIG. 1. In other words, the flow rates decrease from right to left in FIG. 1, and the diameter of the funnel 110 is stepped down from left to right in FIG. 1. The funnel 110 has two ends including a larger end with a larger cross section on the left and a smaller end with a smaller cross section on the right. The wider end of the funnel 110 is at the input which receives the air output from the CO2 module/sensor 104. The funnel 110 may provide a continuous reduction in diameter or a series of steps to step down the diameter to produce a higher pressure on the smaller end and a lower pressure on the larger end. For the lower flow rate required for the CO2 module/sensor 104, the funnel 110 provides for flow reduction in a flow rate output between the CO2 module/sensor 104 and the pump 130.

The teachings herein are not limited to air as the flowing gas or liquid, as air is used as a representative example. The teachings herein are also not limited to combinations of a CO2 module/sensor 104 and blood pressure subsystem 144, as these types of equipment are used as representative examples. The teachings herein are also not limited to stepping up or down to the left of a pump 130, as one or more differentials may be imposed in a flow path on any side of a pump 130 using step mechanisms such as the funnel 110 in FIG. 1. Additionally, while typical values are attributed as requirements for the CO2 module/sensor 104 and the blood pressure subsystem 144, the flow rates may change such as based on differences between different blood pressure cuffs and when a subject is taking a blood pressure cuff off.

The funnel 110 has a diameter that varies along the flow path so as to provide the flow path with a first flow rate and a first pressure for air flowing to the CO2 module/sensor 104 on a first side of the funnel 110 and so as to provide the flow path with a second flow rate and a second pressure for air flowing to the blood pressure subsystem 144 on a second side of the funnel 110 in the flow path opposite to the first side of the funnel 110 in the flow path. The first side of the funnel 110 may be to the left along the flow path in FIG. 1 and the second side of the funnel 110 may be to the right along the flow path in FIG. 1.

The air output from the narrower end of the funnel 110 is passed to the 3-way valve 115. The 3-way valve 115 outputs CO2 exhaust to the outside of the system 100, and filtered air passed from the funnel 110 to the Y fitting 120. The Y fitting 120 allows entry for ambient air flow from the outside for the higher flow rate that the blood pressure subsystem 144 requires.

The Y fitting 120 mixes the filtered air from the 3-way valve and the ambient air from the outside, and passes the mixed air to the input filter 122. The input filter 122 may comprise a second input filter. The input filter 122 filters the mixed air, and passes the filtered air to the check valve 125. The check valve 125 is a one-way mechanism to pass air from the input filter 122 to the pump 130, without allowing any reverse flow from the pump 130.

The pump 130 pumps air into the manifold-valve system 140. The manifold-valve system 140 outputs residual air and pressurizes the blood pressure subsystem 144. The manifold-valve system 140 may include multiple pipes with different diameters, to step up or step down pressure for the blood pressure subsystem 144. The manifold-valve system 140 may comprise a solid assembly with an internal chamber and passages for air to pass into and be diverted to different locations such as the blood pressure subsystem 144, a release valve, a pressure sensor, or other valves/sensors if required. The manifold-valve system 140 may alternatively comprise a set of one or more hoses and one or more Y fittings for air to pass into and be diverted to different locations.

The blood pressure subsystem 144 is an example of a second subsystem, and may include a blood pressure cuff that is inflatable and deflatable. The blood pressure cuff may be inflated based on pressure provided by the pump 130 through the manifold-valve system 140. The blood pressure subsystem 144 may require a high flow rate of 0.6 L/min and a high pressure of 300 mmHg. The blood pressure subsystem 144 may require a pressure that is approximately 4 times higher than the pressure for the CO2 module/sensor 104.

As shown in FIG. 1, the system includes the pump 130 and a flow path that flows to the CO2 module/sensor 104 as a first subsystem that requires a first flow rate and a first pressure, and to the blood pressure subsystem 144 as a second subsystem that requires a second flow rate different than the first flow rate and that requires a second pressure different than the first pressure. The system 100 may be provided in a room or bay in an intensive care unit (ICU) or emergency room (E/R) at a hospital. For example, a subject may be monitored continuously for carbon dioxide while in the room or bay via an input with the input filter 102 at or around the nose or mouth. The CO2 module/sensor 104 may be connected and provide readings to a patient monitor (not shown) in addition to providing the output filtered air to the funnel 110. The subject may also be monitored periodically for blood pressure readings while in the room or bay via the blood pressure subsystem 144 at or around an arm. The blood pressure subsystem 144 may also be connected to and provide readings to a patient monitor (not shown). The pump 130 and some other elements may be provided under a bed or against a wall behind a bed in the room or bay. In some embodiments, the pump 130 and other elements may be provided in a roll cart monitoring system, or may be mounted on a wall. In some embodiments, the system 100 may be provided as a compact handheld system that is transportable.

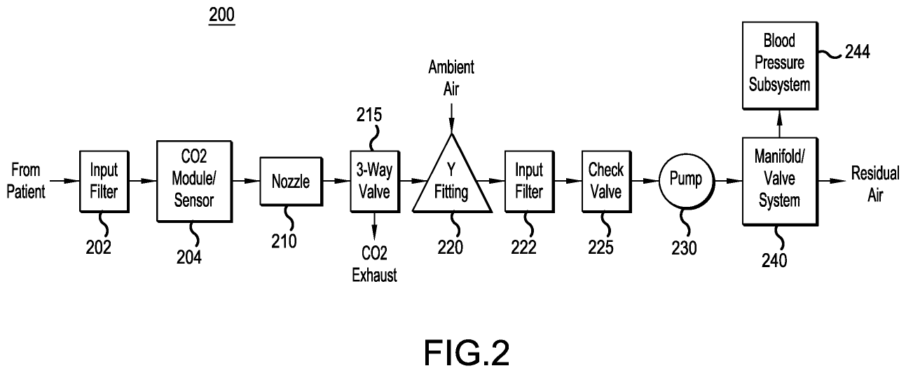

FIG. 2 illustrates another integrated multi-use pump system, in accordance with a representative embodiment.

The system 200 includes an input filter 202, a CO2 module/sensor 204, a nozzle 210, a 3-way valve 215, a Y fitting 220, an input filter 222, a check valve 225, a pump 230, a manifold-valve system 240, and a blood pressure subsystem 244.

The system 200 in FIG. 2 is simplified and includes the CO2 module/sensor 204 as a capnography subsystem requiring patient exhaled air to be pulled through a chamber for CO2 measurement. This air is pulled though the CO2 module/sensor 204 via the pump 230 on the far side of the system 200. At the same time, the blood pressure subsystem 244 may include a non-invasive blood pressure cuff which requires air to be pushed into a volume to inflate the blood pressure cuff. The pump 230 is used to push air to the blood pressure subsystem 244 and to pull air for the CO2 module/sensor 204.

The system 200 includes the input filter 202 between the patient and the CO2 module/sensor 204 to filter the patient exhaled air. The input filter 202 may comprise a first input filter. The input filter 202 passes filtered air to the CO2 module/sensor 204. The CO2 module/sensor 204 is an example of a first subsystem, and specifically comprises a capnography subsystem in embodiments based on FIG. 2. The CO2 module/sensor 204 may require a low flow rate of 60 mL/min and ambient pressure.

The CO2 module/sensor 204 outputs air through a nozzle 210. The nozzle 210 is an example of a flow rate step down. The flow rate output of the air from the CO2 module/sensor 204 is stepped up from the left of the nozzle 210 to the right of the nozzle 210 in FIG. 2. The diameter of the nozzle 210 is stepped down through the nozzle 210 from left to right in FIG. 2. In other words, the flow rates decrease from right to left in FIG. 2, and the diameter of the nozzle 210 is stepped down from left to right in FIG. 2. The nozzle 210 has two ends including a larger end with a larger cross section and a smaller end with a smaller cross section. The wider end of the nozzle 210 is at the input which receives the air output from the CO2 module/sensor 204. The nozzle 210 may provide a continuous reduction in diameter or a series of steps to step down the diameter to produce a higher pressure on the smaller end and a lower pressure on the larger end. For the lower flow rate that the CO2 module/sensor 204 requires, the nozzle 210 provides for reduction in flow rate output between the CO2 module/sensor 204 and the pump 230.

The teachings herein are not limited to a system that flows air, to the use of a nozzle 210 as a flow rate step down mechanism, or to the number and types of subsystems shown in FIG. 2. The teachings herein are also not limited to stepping up or down to the left or right of a pump 230, as one or more differentials may be imposed in a flow path on any side of a pump 230 using step mechanisms such as the nozzle 210 in FIG. 2. Additionally, while typical values are attributed as requirements for the CO2 module/sensor 204 and the blood pressure subsystem 244, the local flow rates may change such as based on differences between different blood pressure cuffs and when a subject is taking a blood pressure cuff off.

The nozzle 210 has a diameter that varies along the flow path so as to provide the flow path with a first flow rate and a first pressure for air flowing to the CO2 module/sensor 104 on a first side of the nozzle 210 and so as to provide the flow path with a second flow rate and a second pressure for air flowing to the blood pressure subsystem 144 on a second side of the nozzle 210 in the flow path opposite to the first side of the nozzle 210 in the flow path. The first side of the nozzle 210 may be to the left along the flow path in FIG. 2 and the second side of the nozzle 210 may be to the right along the flow path in FIG. 2.

The air output from the narrower end of the nozzle 210 is passed to the 3-way valve 215. The 3-way valve 215 outputs CO2 exhaust to the outside of the system 200, and filtered air passed from the nozzle 210 to the Y fitting 220. The Y fitting 220 allows ambient air flow from the outside for the higher flow rate that the blood pressure subsystem 244 requires. The Y fitting 220 mixes the filtered air from the 3-way valve and the ambient air from the outside and passes the mixed air to the input filter 222. The input filter 222 may comprise a second input filter. The input filter 222 filters the mixed air, and passes the filtered air to the check valve 125. The check valve 225 is a one-way mechanism to pass air from the input filter 222 to the pump 230, without allowing any reverse flow from the pump 230.

The pump 230 pumps air into the manifold-valve system 240. The manifold-valve system outputs residual air and pressurizes the blood pressure subsystem 244. The manifold-valve system 240 may include multiple pipes with different diameters, to step up or step down pressure for the blood pressure subsystem 144. The manifold-valve system 240 may comprise a solid assembly with an internal chamber and passages for air to pass into and be diverted to different locations such as the blood pressure subsystem 244, a release valve, a pressure sensor, or other valves/sensors if required. The manifold-valve system 240 may alternatively comprise a set of one or more hoses and one or more Y fittings for air to pass into and be diverted to different locations.

The blood pressure subsystem 244 is an example of a second subsystem, and may include a blood pressure cuff that is inflatable and deflatable. The blood pressure cuff may be inflated based on pressure provided by the pump 230 through the manifold-valve system 240. The blood pressure subsystem 244 may require a high flow rate of 0.6 L/min and a high pressure of 300 mmHg. The blood pressure subsystem 244 may require a pressure that is more than the pressure for the CO2 module/sensor 204, such as by a multiple of 4 times or 10 times.

As shown in FIG. 2, the system includes the pump 230 and a flow path that flows to the CO2 module/sensor 204 as a first subsystem that requires a first flow rate and a first pressure, and to the blood pressure subsystem 244 as a second subsystem that requires a second flow rate different than the first flow rate and that requires a second pressure different than the first pressure. The system 200 may be provided in a room or bay in an intensive care unit (ICU) or emergency room (E/R) at a hospital. For example, a subject may be monitored continuously for carbon dioxide while in the room or bay via an input with the input filter 202 at or around the nose or mouth. The CO2 module/sensor 204 may be connected and provide readings to a patient monitor (not shown) in addition to providing the output filtered air to the nozzle 210. The subject may also be monitored periodically for blood pressure readings while in the room or bay via the blood pressure subsystem 244 at or around an arm. The blood pressure subsystem 244 may also be connected to and provide readings to a patient monitor (not shown). The pump 230 and some other elements may be provided under a bed or against a wall behind a bed in the room or bay. In some embodiments, the system 200 may be provided as a compact handheld system that is transportable.

In the embodiment of FIG. 2, the nozzle 210 replaces the funnel 110 from the embodiment of FIG. 1. As should be clear, the features shown in elements of the system 100 in FIG. 1 and the system 200 in FIG. 2 are representative of aspects of the teachings herein.

However, integrated multi-use pump systems are not limited to the elements shown or to the medical context, let alone to use with only two subsystems such as capnography subsystems and blood pressure subsystems.

The pump 230 moves air by both push and pull through a flow path in the system 200 for at least the two subsystems that require various different flow rates and pressures.

The flow reduction techniques in the system 200 are implemented using the nozzle 210 in cooperation with other elements. The combined effect of the system 200 using the pump 230 for both subsystems is a reduction in power consumption and a reduction in space used.

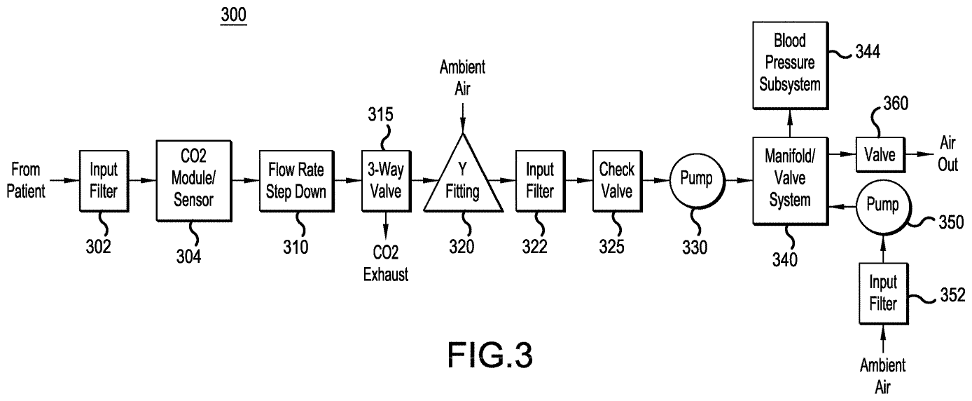

FIG. 3 illustrates another integrated multi-use pump system, in accordance with a representative embodiment.

The system 300 includes an input filter 302, a CO2 module/sensor 304, a flow rate step down 310, a 3-way valve 315, a Y fitting 320, an input filter 322, a check valve 325, a pump 330, a manifold-valve system 340, a blood pressure subsystem 344, a pump 350, an input filter 352 and a valve 360.

The system 300 in FIG. 3 is simplified and includes the CO2 module/sensor 304 as a capnography subsystem requiring patient exhaled air to be pulled through a chamber for CO2 measurement. This air is pulled through the CO2 module/sensor 304 via the pump 330 and the pump 350 on the far side of the system 300. At the same time, the blood pressure subsystem 344 may include a non-invasive blood pressure cuff which requires air to be pushed into a volume to inflate the blood pressure cuff. The pump 330 and the pump 350 are used to push air to the blood pressure subsystem 344 and to pull air for the CO2 module/sensor 304.

The system 300 includes the input filter 302 between the patient and the CO2 module/sensor 304 to filter the patient exhaled air. The input filter 302 may comprise a first input filter. The input filter 302 filters air exhaled from a patient. The input filter 302 passes filtered air to the CO2 module/sensor 304. The CO2 module/sensor 304 is an example of a first subsystem, and specifically comprises a capnography subsystem in embodiments based on FIG. 3. The CO2 module/sensor 304 may require a low flow rate of 60mL/min and ambient pressure.

The CO2 module/sensor 304 outputs air through a flow rate step down 310. The flow rate output of the air from the CO2 module/sensor 304 is stepped up from the left of the flow rate step down 310 to the right of the flow rate step down 310. The diameter of the flow rate step down is stepped down from left to right in FIG. 3. In other words, the flow rates decrease from right to left in FIG. 3, and the diameter of the funnel flow rate step down 310 is stepped down from left to right in FIG. 3. The flow rate step down 310 has two ends including a smaller end with a smaller cross section and a larger end with a larger cross section. The wider end of the flow rate step down 310 is at the input which receives the air output from the CO2 module/sensor 304. The flow rate step down 310 may provide a continuous reduction in diameter or a series of steps to step down the diameter to produce a higher pressure on the smaller end and a lower pressure on the larger end. For the lower flow rate that the CO2 module/sensor 304 requires, the flow rate step down 310 provides for flow reduction between the CO2 module/sensor 304 and the pump 330 and pump 350.

The flow rate step down 310 has a diameter that varies along the flow path so as to provide the flow path with a first flow rate and a first pressure for air flowing to the CO2 module/sensor 104 on a first side of the flow rate step down 310 and so as to provide the flow path with a second flow rate and a second pressure for air flowing to the blood pressure subsystem 144 on a second side of the flow rate step down 310 in the flow path opposite to the first side of the flow rate step down 310 in the flow path. The first side of the flow rate step down 310 may be to the left along the flow path in FIG. 3 and the second side of the flow rate step down 310 may be to the right along the flow path in FIG. 3.

The air output from the narrower end of the flow rate step down 310 is passed to the 3-way valve 315. The Y fitting 320 allows ambient air flow for the higher flow rate that the blood pressure subsystem 344 requires. The 3-way valve 315 outputs CO2 exhaust to the outside of the system 300, and filtered air passed from the flow rate step down 310 to the Y fitting 320. The Y fitting 320 also receives ambient air from the outside. The Y fitting 320 mixes the filtered air from the 3-way valve and the ambient air from the outside and passes the mixed air to the input filter 322. The input filter 322 may comprise a second input filter. The input filter 322 filters the mixed air and passes the filtered air to the check valve 325. The check valve 325 is a one-way mechanism to pass air from the input filter 322 to the pump 330, without allowing any reverse flow from the pump 330.

The pump 330 pumps air into the manifold-valve system 340. The manifold-valve system 340 may comprise a solid assembly with an internal chamber and passages for air to pass into and be diverted to different locations such as the blood pressure subsystem 344, a release valve, a pressure sensor, or other valves/sensors if required. The manifold-valve system 340 may alternatively comprise a set of one or more hoses and one or more Y fittings for air to pass into and be diverted to different locations. The manifold-valve system 340 may include multiple pipes with different diameters, to step up or step down pressure for the blood pressure subsystem 144.

The pump 350 also pumps air into the manifold-valve system 340. The input filter 352 filters ambient air input to the pump 350, and the valve 360 serves as an outlet for air from the manifold-valve system 340. The manifold-valve system outputs residual air and pressurizes the blood pressure subsystem 344. The blood pressure subsystem 344 is an example of a second subsystem and may include a blood pressure cuff that is inflatable and deflatable. The blood pressure cuff may be inflated based on pressure provided by the pump 330 through the manifold-valve system 340.

As shown in FIG. 3, the system includes the pump 330 and a flow path that flows to the CO2 module/sensor 304 as a first subsystem that requires a first flow rate and a first pressure, and to the blood pressure subsystem 344 as a second subsystem that requires a second flow rate different than the first flow rate and that requires a second pressure different than the first pressure. The system 300 may be provided in a room or bay in an intensive care unit (ICU) or emergency room (E/R) at a hospital. For example, a subject may be monitored continuously for carbon dioxide while in the room or bay via an input with the input filter 302 at or around the nose or mouth. The CO2 module/sensor 304 may be connected and provide readings to a patient monitor (not shown) in addition to providing the output filtered air to the flow rate step down 310. The subject may also be monitored periodically for blood pressure readings while in the room or bay via the blood pressure subsystem 344 at or around an arm. The blood pressure subsystem 344 may also be connected to and provide readings to a patient monitor (not shown). The pump 330 and some other elements may be provided under a bed or against a wall behind a bed in the room or bay. In some embodiments, the pump 330, the pump 350 and other elements may be provided in a roll cart monitoring system, or may be mounted on a wall. In some embodiments, the system 300 may be provided as a compact handheld system that is transportable.

In the embodiment of FIG. 3, the flow rate step down 310 replaces the funnel 110 from the embodiment of FIG. 1 and the nozzle 210 of FIG. 2 and serves as a damper system. Additionally, the pump 350 is a second pump which is used to provide pressure to the blood pressure subsystem 344 through the manifold-valve system 340. The input filter 352 filters ambient air provided to the pump 350. The valve 360 outputs air from the manifold-valve system 340. As should be clear, the features shown in elements of the system 100 in FIG. 1, the system 200 in FIG. 2 and the system 300 in FIG. 3 are representative of aspects of the teachings herein. However, integrated multi-use pump systems are not limited to the elements shown or to the medical context let alone to use with only two systems such as capnography subsystems and blood pressure systems.

The pump 330 and the pump 350 move air by both push and pull through a flow path in the system 300 for at least the two subsystems that require various different flow rates and pressures. The flow reduction techniques in the system 300 are implemented using the flow rate step down 310 in cooperation with other elements. The combined effect of the system 300 using the pump 330 and the pump 350 for both subsystems is a reduction in power consumption and a reduction in space used.

While only one pump is shown in the systems of FIG. 1 and FIG. 2, and two pumps are shown in the system of FIG. 3, the number of pumps that may be used in an integrated pumping system for multiple application subsystems is not limited to one or two, and instead may be more than two. Additionally, while two subsystems are shown in the systems of FIG. 1, FIG. 2 and FIG. 3, the number of subsystems with varying pressure and flow rate requirements is not limited to two, and instead may be more than two.

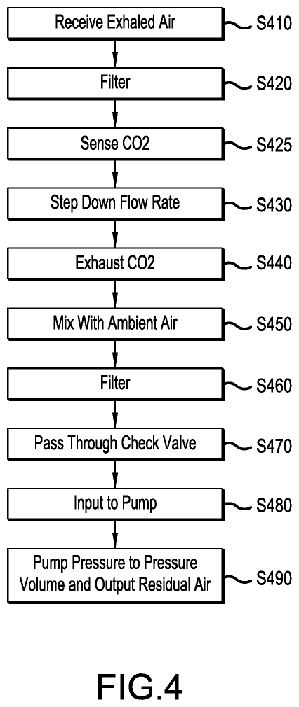

FIG. 4 illustrates a method of operation for an integrated multi-use pump system, in accordance with another representative embodiment.

At S410, exhaled air is received. The exhaled air may be received from a patient via a mouthpiece for capnography.

At S420, the exhaled air is filtered. The filtering may be by the input filter 102, the input filter 202 or the input filter 302.

After filtering at S420, the filtered air is tested by sensing in a chamber at S425 such as by the CO2 module/sensor 104, the CO2 module/sensor 204 or the CO2 module/sensor 304.

At S430, the flow rate of the filtered exhaled air is stepped down. The flow rate output from the CO2 modules/sensors of the filtered air may be stepped down by the funnel 110, the nozzle 210 or the flow rate step down 310. That is, the flow rate may be stepped down by passing the filtered exhaled air through a conduit with a varied diameter along a length of the flow path. The diameter may be larger on a first side of the conduit and smaller on a second side of the conduit opposite to the first side.

At S440, CO2 from the stepped down air is exhausted. The CO2 may be exhausted from the 3-way valve 115, the 3-way valve 215 or the 3-way valve 315.

At S450, the stepped down air is mixed with ambient air. The mixing may be performed at the Y fitting 120, the Y fitting 220, or the Y fitting 320.

At S460, the mixed air is filtered. The filtering may be performed by the input filter 122, the input filter 222 or the input filter 322.

At S470, the filtered mixed air is passed through a check valve. The check valve may be implemented by the check valve 125, the check valve 225 or the check valve 325. The check valve ensures that the air flows in one direction, i.e., towards the pump from the CO2 module sensor in each of FIG. 1, FIG. 2 and FIG. 3.

At S480, the air passed through the check valve is input to a pump. The air may be input to the pump 130, to the pump 230 or to the pump 330.

At S490, the pump pressure is provided to a pressure volume, and residual air is output. The pump pressure may be provided via a manifold-valve system 140 to the blood pressure subsystem 144, via a manifold-valve system 240 to the blood pressure subsystem 244, or via the manifold-valve system 340 to the blood pressure subsystem 344.

Additionally, in embodiments such as embodiments based on FIG. 3, one or more additional pump such as the pump 350 may be used to provide pressure to a second subsystem such as the blood pressure subsystem 344 via the manifold-valve system 340. More than 2 pumps may exist in an integrated multi-use pump system. For example, 4 pumps may be used together, individually, or in subgroups of more than 1 and less than 4 to provide pressure to a second subsystem.

Although the teachings herein primarily use the example of subsystems for non-invasive blood pressure and capnography, the integrated multi-use pump system is not limited to these example subsystems. For example, an integrated multi-use pump system may be used for a non-invasive blood pressure subsystem and an anesthetic subsystem, so long as proper filtering is put in place. Additionally, while system 100, system 200 and system 300 are described in a context which suggests that the first subsystem and the second subsystem are used simultaneously, the first subsystem and the second subsystem are used independently and may be used at different times. In some embodiments, a first subsystem such as the CO2 module/sensor 104 may be used continuously for an anesthetized patient, and a second subsystem such as the blood pressure subsystem 144 may be used intermittently for the same anesthetized patient.

Moreover, there is no specific requirement that only one subject use the different subsystems of a single integrated multi-use pump system. For example, so long as proper filtering is provided, even in the medical context multiple different subjects may use the same pump or set of pumps for different subsystems such as blood pressure measurements and CO2 monitoring. However, as should be clear in the context of the descriptions herein, the same pump or set of pumps is used to pull air on one side and push air on another.

Accordingly, the integrated multi-use pump system teachings provided herein enable integration of a multi-use pump system which includes one or more pump(s) and a flow path. The flow path flows air to a first subsystem that requires a first flow rate and a first pressure, and to a second subsystem that requires a second flow rate different than the first flow rate and that requires a second pressure different than the first pressure.

Although integrated multi-use pump system has been described with reference to several exemplary embodiments, it is understood that the words that have been used are words of description and illustration, rather than words of limitation. Changes may be made within the purview of the appended claims, as presently stated and as amended, without departing from the scope and spirit of integrated multi-use pump system in its aspects. Although integrated multi-use pump system has been described with reference to particular means, materials and embodiments, integrated multi-use pump system is not intended to be limited to the particulars disclosed; rather integrated multi-use pump system extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept.

Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. An integrated multi-use pump system, comprising:
at least one pump; and
a flow path that flows air to a first subsystem that requires a first flow rate and a first pressure, and to a second subsystem that requires a second flow rate different than the first flow rate and that requires a second pressure different than the first pressure.

2. The integrated multi-use pump system of claim 1,
wherein the at least one pump is centralized and the integrated multi-use pump system is used to simultaneously operate the first subsystem and the second subsystem.

3. The integrated multi-use pump system of claim 1, further comprising:
the first subsystem, wherein the first subsystem comprises a capnography subsystem.
the second subsystem, wherein the second subsystem comprises a blood pressure subsystem.

4. The integrated multi-use pump system of claim 1,
wherein the flow path is used to push air to the first subsystem and to pull air from the second subsystem.

5. The integrated multi-use pump system of claim 1, wherein the flow path is configured to modify a flow rate output from the first subsystem in the first flow rate and into the second flow rate.

6. The integrated multi-use pump system of claim 5, wherein the flow path includes at least one funnel to reduce the flow rate output from the pump.

7. The integrated multi-use pump system of claim 5, wherein the flow path includes at least one nozzle to reduce the flow rate output from the pump.

8. The integrated multi-use pump system of claim 3, wherein the capnography subsystem pulls air exhaled from a patient through a chamber for CO2 measurement, and the blood pressure subsystem inflates a cuff.

9. The integrated multi-use pump system of claim 8, further comprising:
a first filter between the pump and the capnography subsystem to filter the air exhaled from the patient; and
a fitting to supplement the air exhaled from the patient with ambient air.

10. The integrated multi-use pump system of claim 9, further comprising:
a check valve to restrict air flow from the pump to the fitting.

11. The integrated multi-use pump system of claim 10, further comprising:
a manifold-valve system between the pump and the blood pressure subsystem.

12. The integrated multi-use pump system of claim 8, further comprising:
a damper system for reducing the flow rate output from the capnography subsystem to the pump.

13. The integrated multi-use pump system of claim 1, wherein the at least one pump comprises a plurality of pumps.

14. A method of operating an integrated multi-use pump system, comprising:
receiving and filtering exhaled air;
stepping down a flow rate of the exhaled air;
mixing the exhaled air with ambient air to obtain an air mixture;
filtering the air mixture;
passing the air mixture through a check valve;
inputting the air mixture to a pump; and
applying pressure to a pressure to a pressure volume using the air mixture.

15. The method of claim 14, wherein the exhaled air is received in a flow path that flows air to a first subsystem that requires a first flow rate and a first pressure, and the pressure volume is pressurized to a second pressure different than the first pressure.

16. An integrated multi-use pump system, comprising:
at least one pump; and
a conduit with a diameter that varies along a flow path so as to provide the flow path with a first flow rate and a first pressure for air flowing to a first subsystem on a first side of the conduit in the flow path and so as to provide the flow path with a second flow rate and a second pressure for air flowing to a second subsystem on a second side of the conduit in the flow path opposite to the first side of the conduit in the flow path.
